(19) United States
(12) Patent Application Publication
(10) Pub. No.: US 2020/0209262 A1
(43) Pub. Date: Jul. 2, 2020

(54) BIOMARKERS FOR WOUND HEALING

(71) Applicants: MCMASTER UNIVERSITY, Hamilton (CA); UNIVERSITY OF WESTERN AUSTRALIA, Crawley (AU)

(72) Inventors: Michael Stacey, Hamilton (CA); Steven Phillips, Brantford (CA); Jillian Marlene Swain, Crawley (AU)

(21) Appl. No.: 16/622,891

(22) PCT Filed: Jun. 14, 2018
(86) PCT No.: PCT/CA2018/050720

§ 371 Date: Dec. 13, 2019

Related U.S. Application Data
(60) Provisional application No. 62/519,478, filed on Jun. 14, 2017.

(51) Int. Cl. G01N 33/68

(52) U.S. Cl. CPC G01N 33/6893; G01N 33/6863; G01N 2800/52; G01N 2333/96494; G01N 2800/20; G01N 2333/535

(57) ABSTRACT

A method for determining healing status of a wound is provided comprising: i) quantifying the expression level of GM-CSF or MMP-13, and optionally one or more additional biomarkers, in a wound tissue sample or wound fluid sample from a wound of a mammalian subject; and ii) comparing the expression level of GM-CSF or MMP-13 of the wound tissue sample or wound fluid sample to a threshold level and determining that the wound is non-healing if the level of GM-CSF or MMP-13 exceeds the threshold level.

### FIELD OF THE INVENTION

The present application relates to the field of wound healing, to methods for monitoring the status and rate of wound healing and to methods for identifying agents that can facilitate the repair and healing of wounds, particularly chronic wounds. The present invention is also directed to a kit for assessing wound status.

### BACKGROUND OF THE INVENTION

Chronic wounds due to venous disease or diabetes represent a significant healthcare burden due to high costs associated with treating these complications. It has been reported that venous leg ulcers (VLU) occur in 2% of patients over the age of 60, and up to 15% of patients with diabetes will experience a diabetic foot ulcer (DFU). These wounds often exhibit impairments in healing, as 24% of VLU are present for a period of longer than 1 year. In addition, once an ulcer has healed they show high rates of recurrence, with 50% of DFU recurring within 3 years of initial healing and 70% of healed VLU recurring. The combination of impaired healing and high recurrence leads to a decrease in quality of life for patients suffering from these wounds.

The chronic wound environment is drastically different to that of acute wounds. Chronic wounds exhibit an excessive, sustained inflammatory phase which is characterized by increases in inflammatory cytokines such as tumor necrosis factor alpha (TNF-α) and various interleukins, as well as increased protease activity including matrix metalloproteases (MMP) and neutrophil elastase. The increased level of proteases break down endogenous growth factors and degrade the extracellular matrix, impairing the ability of the wound to heal.

A variety of treatment methods have been utilized to treat such wounds including wound dressings, topical medications, surgical intervention, compression bandaging and pressure offloading. Currently, the leading approach to determine if a wound is healing includes repeated surface area measurements to evaluate if the wound is decreasing in size over time. This approach is somewhat problematic as it requires waiting several weeks in order to verify if a wound is decreasing in size, resulting in lost time for re-evaluating the wound and considering an alternate treatment regimen to improve the outcome of the wound.

It would be desirable, thus, to identify one or more biomarkers useful to determine the healing status of a wound, and whether the wound is on a healing or non-healing trajectory, to allow caregivers to adapt their treatment approach in a more timely fashion.

### SUMMARY OF THE INVENTION

The present invention is directed to the diagnosis of wounds and specifically to the diagnosis of wound status. The term “wound status” refers to the condition of a wound and whether or not the wound is a healing wound or a non-healing wound, and therefore a chronic wound.

In one aspect of the present invention, wound status is determined by measuring the level of GM-CSF in a wound. The level of GM-CSF is then compared to a GM-CSF threshold level and the wound is determined to be non-healing if the GM-CSF protein level is determined to exceed the predetermined threshold level.

In another aspect of the present invention, wound status is determined by measuring MMP-13 levels in a wound. The level of MMP-13 is then compared to a MMP-13 threshold level and the wound is determined to be non-healing if the MMP-13 protein level exceeds the predetermined threshold level.

In an embodiment, the level of expression of at least one biomarker additional to GM-CSF or MMP-13 is measured in a wound sample and compared to a predetermined threshold level. The additional biomarker is selected from the group consisting of GM-CSF, MMP-13, albumin, calcium, eotaxin, glucose, ICAM-1, IL-6, IL-16, MCP-1, MIP-1a, PDGF-BB and TIMP-4.

In another aspect of the invention, a kit is provided comprising at least one reactant that specifically reacts with GM-CSF and/or MMP-13, and optionally, one or more additional reactants that specifically react with a second target biomarker selected from the group consisting of GM-CSF, MMP-13, albumin, calcium, eotaxin, glucose, ICAM-1, IL-6, IL-16, MCP-1, MIP-1a, PDGF-BB and TIMP-4.

In a further aspect of the present invention, a kit is provided for use to determine healing status of a wound. The kit comprises a plurality of implements configured to collect or absorb thereon a sample of fluid from a wound, and a panel on which is bound at least one reactant specific for a biomarker selected from GM-CSF and MMP-13 which yields a detectable signal that corresponds with the level of said biomarker.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

These and other aspects of the invention are described in the detailed description that follows by reference to the following figures.

### BRIEF DESCRIPTION OF THE FIGURES

FIG. 1 graphically illustrates the healing, non-healing, and indeterminate wound time points for a patient.

FIG. 2 graphically illustrates the change in wound area vs. GM-CSF levels. Points to the left of zero on the x-axis represent healing weeks while points to the right of zero on the x-axis represent non-healing weeks.

FIG. 3 graphically illustrates the change in wound area vs. MMP-13 levels. Points to the left of zero on the x-axis represent healing weeks while points to the right of zero on the x-axis represent non-healing weeks.

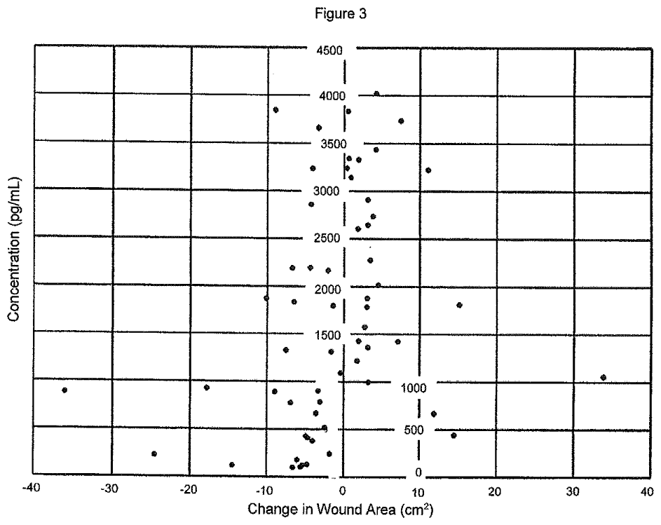

### DETAILED DESCRIPTION OF THE INVENTION

A method for determining wound healing status of a wound in a mammalian subject is provided comprising: i) quantifying the expression level of GM-CSF or MMP-13 in a tissue or fluid sample from the wound; and ii) comparing the expression level of GM-CSF or MMP-13 in the wound tissue or fluid sample to a predetermined threshold level and determining that the wound is non-healing if the level of GM-CSF or MMP-13 exceeds the threshold level.

The term “wound” is used herein to refer to any type of wound, but has particular use in determining wound healing status in a chronic wound in which there is a failure to heal within an expected timeframe. Common wounds include infectious wounds that may result from bacterial, fungal or viral infection which will exhibit a longer healing time in the absence of medication targeting the infectious agent; ischemic wounds to which there is an insufficient blood supply limiting both oxygen and nutrient flow to the wound, and wound healing accordingly; radiation poisoning wounds by various sources of radiation (e.g. gamma rays, x-rays or exposure to radioactive materials) which can weaken the immune system and delay healing; surgical wounds, healing of which may be delayed if sufficient blood supply or care are inadequate; and ulcers, such as arterial ulcers, venous ulcers, pressure ulcers and diabetic ulcers.

The term “GM-CSF” refers to granulocyte macrophage colony stimulating factor or colony stimulating factor-2. GM-CSF is a monomeric glycoprotein, secreted by macrophages, T cells, mast cells, natural killer cells, endothelial cells and fibroblasts, that functions as a cytokine. As used herein, GM-CSF encompasses full-length mammalian GM-CSF, including human and functionally equivalent variants thereof such as non-human GM-CSF. Functionally equivalent variants of full-length GM-CSF encompass full-length GM-CSF orthologs, isoforms and variants thereof which may incorporate alterations, such as, but not limited to, minor amino acid alternations such as deletions, additions or substitutions, which do not significantly adversely affect GM-CSF activity. Transcript sequences of various forms of full-length GM-CSF are known and readily accessible on sequence databases, such as NCBI, by reference to nucleotide accession nos., e.g. human GM-CSF (NM_000758), mouse GM-CSF (NM_009969) and canine GM-CSF (NM_001003245.1). GM-CSF amino acid sequences are also known such as human (NP_000749), mouse (NP_034099) and canine (NP_001003245.1).

The term “MMP-13” refers to matrix metalloprotease-13 or collagenase 3, an enzyme of the matrix metalloproteinase (MMP) family involved in the breakdown of extracellular matrix in normal physiological processes. As used herein, MMP-13 encompasses full-length mammalian MMP-13, including human and functionally equivalent variants thereof such as non-human MMP-13. Functionally equivalent variants of full-length MMP-13 encompass full-length MMP-13 orthologs, isoforms and variants thereof which may incorporate alterations, such as, but not limited to, minor amino acid alternations such as deletions, additions or substitutions, which do not significantly adversely affect MMP-13 activity. Transcript sequences of various forms of full-length MMP-13 are known and readily accessible on sequence databases, such as NCBI, by reference to nucleotide accession nos., e.g. human MMP-13 (NM_002427) and mouse MMP-13 (NM_008607). MMP-13 amino acid sequences are also known such as human (NP_002418) and mouse (NP_032633).

The mammalian subject may be a human or non-human mammal such as a domestic animal (e.g. dog, cat, cow, horse, pig, goat and the like) or a non-domestic animal.

The expression level of GM-CSF or MMP-13 protein is determined in a wound tissue or fluid sample of the mammalian subject. Such samples are obtained using established techniques, for example, by swabbing, blotting, scraping or aspirating fluid or tissue from a wound site.

Once a wound tissue or fluid sample is obtained, the level of the selected biomarker, either transcript level or protein concentration, is detected and quantified within the sample. As one of skill in the art will appreciate, the expression level of the biomarker may be determined using one of several techniques established in the art, including methods of quantifying nucleic acid encoding the target biomarker, such as PCR-based techniques, microarrays, gene expression system, and Northern or Southern blotting techniques, or methods of quantifying the protein biomarker, such as immunoassay (including ELISA), multiplex assays, activity assays, Western blotting, mass spectrometry, high performance liquid chromatography and two-dimensional electrophoresis.

In one embodiment, the expression level of the selected biomarker in a biological sample from a mammal is determined based on the levels of nucleic acid (i.e. DNA or mRNA transcript) encoding the target protein biomarker in the biological sample. Methods of determining DNA or mRNA levels are known in the art, and include, for example, PCR-based techniques (such as RT-PCR), and Northern or Southern blotting techniques which generally include the application of gel electrophoresis to isolate the target nucleic acid, followed by hybridization with specific labeled probes. Oligonucleotide probes for use in these methods are readily designed based on the known sequences of genes encoding the protein biomarker, as well as the known amino acid sequence of the target biomarker, and may comprise about 15-40 nucleotides, for example, 20-35 nucleotides. Probes that target GM-CSF and/or MMP-13 nucleic acids, thus, are designed to bind to a region of the genomic or transcript nucleic acid sequence encoding these proteins. Software has been developed for this purpose by Roche Life Sciences, Sigma Aldrich, LCG Biosearch Technologies, and others. Suitable labels for use are well-known, and include, for example, fluorescent, chemiluminescent and radioactive labels.

In other embodiments, the expression level of GM-CSF and/or MMP-13 protein in a sample may be measured by immunoassay using an antibody specific to the target protein. The antibody binds to the target protein and bound antibody is quantified by measuring a detectable marker which may be linked to the antibody or other component of the assay, or which may be generated during the assay. Detectable markers may include radioactive, fluorescent, phosphorescent and luminescent (e.g. chemiluminescent or bioluminescent) compounds, dyes, particles such as colloidal gold and enzyme labels.

The term “antibody” is used herein to refer to monoclonal or polyclonal antibodies, or antigen-binding fragments thereof, e.g. an antibody fragment that retains specific binding affinity for the target biomarker. Antibodies to the target biomarkers are generally commercially available. For example, GM-CSF antibodies to various immunogens, including internal, and N- and C-terminal, are commercially available, for example, from Sigma Alderich, Santa Cruz Biotech and AbCam, while MMP-13 antibodies are commercially available from, for example, AbCam and R&D Systems. Examples of GM-CSF antibodies include M1B8, CC5B5, CC1H7 and CC3C12. Antibodies that target antigens across the GM-CSF protein may be used, including antigenic regions such as amino acid regions 21-31, 77-94, 110-127 and others. MMP-13 antibodies that target antigens across the protein may also be used, including antibodies to both N- and C-terminal regions, and internal regions in between. As one of skill in the art will appreciate, antibodies to the target proteins may also be raised using techniques conventional in the art. For example, antibodies may be made by injecting a non-human host animal, e.g. a mouse or rabbit, with the antigen (target protein or immunogenic fragment thereof), and then isolating antibody from a biological sample taken from the host animal

Different types of immunoassay may be used to determine expression level of target proteins, including indirect immunoassay in which the protein is non-specifically immobilized on a surface; sandwich immunoassay in which the protein is specifically immobilized on a surface by linkage to a capture antibody bound to the surface; competitive binding immunoassay in which a sample is first combined with a known quantity of antibody to bind the target protein in the sample, and then the sample is exposed to immobilized target protein which competes with the sample to bind any unbound antibody. To the immobilized protein/antibody is added a detectably-labeled secondary antibody that detects the amount of immobilized primary antibody, thereby revealing the inverse of the amount of target protein in the sample.

A preferred immunoassay for use to determine expression levels of target protein in a sample is an ELISA (Enzyme Linked ImmunoSorbent Assay) or Enzyme ImmunoAssay (EIA). To determine the level or concentration of the target protein using ELISA, the target protein to be analyzed is generally immobilized, for example, on a solid adherent support, such as a microtiter plate, polystyrene beads, nitrocellulose, cellulose acetate, glass fibers and other suitable porous polymers, which is pretreated with an appropriate ligand for the target, which is then complexed with a specific reactant or ligand such as an antibody which is itself linked (either before or following formation of the complex) to an indicator, such as an enzyme. Detection may then be accomplished by incubating this enzyme-complex with a substrate for the enzyme that yields a detectable product. The indicator may be linked directly to the reactant (e.g. antibody) or may be linked via another entity, such as a secondary antibody that recognizes the first or primary antibody. Alternatively, the linker may be a protein such as streptavidin if the primary antibody is biotin-labeled. Examples of suitable enzymes for use as an indicator include, but are not limited to, horseradish peroxidase (HRP), alkaline phosphatase (AP), ß-galactosidase, acetylcholinesterase and catalase. A large selection of substrates is available for performing the ELISA with these indicator enzymes. As one of skill in the art will appreciate, the substrate will vary with the enzyme utilized. Useful substrates also depend on the level of detection required and the detection instrumentation used, e.g. spectrophotometer, fluorometer or luminometer. Substrates for HRP include 3,3′,5,5′-Tetramethylbenzidine (TMB), 3,3′-Diaminobenzidine (DAB) and 2,2′-azino-bis(3-ethylbenzothiazoline-6-sulphonic acid) (ABTS). Substrates for AP include para-Nitrophenylphosphates. Substrates for ß-galactosidase include β-galactosides; the substrate for acetylcholinesterase is acetylcholine, and the substrate for catalase is hydrogen peroxide.

As will be appreciated by one of skill in the art, assay methods which target the activity of a target protein may also be utilized to determine the expression level thereof in a sample, including for example, ligand-binding assays.

The expression level of GM-CSF and/or MMP-13 in a given sample may be analyzed individually or together using, for example, biochip array technology. Generally, biochip arrays provide a means to simultaneously determine the level of multiple biomarkers in a given sample. These arrays may utilize ELISA technology and, thus, the biochip may be modified to incorporate capture antibodies for each target at pre-defined sites on the surface.

The expression level of GM-CSF and/or MMP-13 is quantified in the wound sample and compared to a predetermined threshold level to determine whether the wound is healing or non-healing. If the presence of GM-CSF is determined in the sample, then the level of GM-CSF is compared to the threshold level of GM-CSF and if it exceeds the threshold level, then the wound is determined to be non-healing. The threshold level is the level of a given protein in a normal tissue or fluid sample (i.e. non-wound tissue or fluid) from the mammal. Generally, the level of GM-CSF in a normal sample (i.e. the threshold level) is in the range of about 15-60 pg/ml, for example, 20-40 pg/ml, 25-35 pg/ml, 28-32 pg/ml or 29-30 pg/ml (such as 29.5 pg/ml). If the level of MMP-13 is determined in the sample, then it is compared to the threshold level of MMP-13 and if it exceeds the threshold level, then the wound is determined to be non-healing. Generally, the level of MMP-13 in a normal sample (i.e. the threshold level) is in the range of about 800-1000 pg/ml, for example, 900-980 pg/ml, 925-975 pg/ml, 950-970 pg/ml or 955-965 pg/ml (such as 962.16 pg/ml). If both GM-CSF and MMP-13 expression levels are determined in a wound, and both exceed the corresponding threshold levels, then the wound is non-healing. If expression levels of both GM-CSF and MMP-13 expression are below the corresponding threshold levels, then the wound is a healing wound.

The level of expression of one or more additional biomarkers may optionally be determined in a wound sample to further confirm the healing status of the wound. Such additional biomarkers include one or more of: albumin, calcium, eotaxin-1, glucose, ICAM-1 (also known as Intercellular Adhesion Molecule 1 or CD54), IL-6, IL-16, MCP-1 (also known as monocyte chemoattractant protein 1 or CCL2), MIP-1α (also known as macrophage inflammatory protein 1-alpha or CCL3), PDGF-BB (platelet-derived growth factor with two B subunits) and TIMP-4 (Metalloproteinase inhibitor 4). Sequence information for these biomarkers is readily available on various sequence databases such as NCBI (National Center for Biotechnology Information).

The level of expression of the one or more additional biomarkers is measured and compared to a predetermined threshold level for that biomarker. The levels of additional protein biomarkers may be measured as described above, e.g. using a suitable immunoassay. Levels of non-protein biomarkers may be determined using methods suitable for their detection. For example, the level of glucose may be determined using enzymatic methods to yield a detectable product (e.g. employing the glucose oxidase, hexokinase or glucose dehydrogenase) or using oxidation/reduction methods such as the p-bromoaniline method (e.g. glucose is converted to furfurol by heating with acetic acid and then combined with p-bromoaniline to yield a complex detectable at 380 nm). A fluorogenic calcium-binding dye may be used, such as an acetoxymethyl (AM) ester, to detect calcium levels.

The threshold levels vary for each biomarker. In addition, while determination of a level of biomarker that is lower than the threshold level is indicative of wound healing in some biomarkers, for other biomarkers the determination of a biomarker level that is higher than the threshold level is indicative of wound healing. Thus, in addition to GM-CSF and MMP-13, levels of the biomarkers, ICAM-1, IL-16 and MIP-1α which are lower than their threshold levels are indicative of wound healing (or levels higher than threshold levels are indicative of non-healing in a wound). Threshold level for ICAM-1 is in the range of about 12×10⁴-14×10⁴ pg/ml, e.g. about 13×10⁴ pg/ml (such as 131405.14 pg/ml). Threshold level for IL-16 is in the range of about 1200-1500 pg/ml or 1400-1500 pg/ml (such as 1449.88 pg/ml). Threshold level for MIP-1α is in the range of about 13000-15000 pg/ml or 14000-15000 pg/ml (such as 14552.18 pg/ml).

On the other hand, biomarkers in which biomarker levels that are higher than the threshold level are indicative of wound healing (or biomarker levels lower than threshold levels are indicative of non-healing in a wound) include, albumin, calcium, eotaxin-1, glucose, IL-6, MCP-1, PDGF-BB and TIMP-4. Threshold level for albumin is in the range of about 5-15 g/L or 8-12 g/L (such as 10.45 g/L). Threshold level for calcium is in the range of about 0.8-1.2 mmol/L or 0.9-1.1 mmol/L (such as 1.03 mmol/L). Threshold level for eotaxin-1 is in the range of about 300-400 pg/ml (such as 343.04 pg/ml). Threshold level for glucose is in the range of about 0.8-1.3 mmol/L or 0.9-1.25 mmol/L (such as 1.185 mmol/L). Threshold level for IL-6 is in the range of about 3000-4000 pg/ml (such as 3499.17 pg/ml). Threshold level for MCP-1 is in the range of about 1700-1800 pg/ml (such as 1784.92 pg/ml). Threshold level for PDGF-BB is in the range of about 25-80 pg/ml or 40-60 pg/ml (such as 55.54 pg/ml). Threshold level for TIMP-4 is in the range of about 650-800 pg/ml or 700-750 pg/ml (such as 738.21 pg/ml).

In another aspect of the invention, a method of monitoring the treatment of a chronic wound in a mammalian subject is provided. The method comprises: (a) determining the level of GM-CSF or MMP-13, and optionally one or more of GM-CSF, MMP-13, albumin, calcium, eotaxin-1, glucose, ICAM-1, IL-6, IL-16, MCP-1, MIP-1α, PDGF-BB and TIMP-4, in a first wound sample from the subject to create a first biomarker profile; (b) treating the chronic wound; (c) determining the level of GM-CSF or MMP-13, and optionally one or more of GM-CSF, MMP-13, albumin, calcium, eotaxin-1, glucose, ICAM-1, IL-6, IL-16, MCP-1, MIP-1a, PDGF-BB and TIMP-4, in a second wound sample from the subject subsequent to treatment of the chronic wound to create a second biomarker profile; and (d) comparing the first and second biomarker profiles, wherein a decrease in the level of at least one of GM-CSF or MMP-13 is indicative of wound healing and indicates that the treatment is effective.

In one method of monitoring effectiveness of the treatment of a chronic wound, a first wound tissue sample or wound fluid sample is obtained from the wound and the levels GM-CSF or MMP-13, and optionally one or more of additional biomarkers selected from GM-CSF, MMP-13, albumin, calcium, eotaxin-1, glucose, ICAM-1, IL-6, IL-16, MCP-1, PDGF-BB and TIMP-4 is determined as described above to create a first biomarker profile. The wound is then treated, and following a sufficient period of time, i.e. a period of time sufficient for the treatment to have an effect, the levels of GM-CSF or MMP-13 and optionally one or more of the additional biomarkers are determined to provide a second biomarker profile. The first and second biomarker profiles are compared to a pre-defined value, e.g. to threshold levels of the biomarkers, and a change in biomarker levels indicative of wound healing indicates that the treatment is effective.

Wound treatments may include treatment with an antimicrobial agent (e.g. antibacterial, antifungal or antiviral agent), or treatment with an anti-inflammatory agent such as a non-steroidal anti-inflammatory agent (NSAID) such as ibuprofen, aspirin or naproxen, or non-NSAID agents such as acetaminophen. In some cases, more advanced therapies such as the use of growth factors (e.g. EGF, FGF), extracellular matrices (ECMs), engineered skin, stem cell therapy, negative pressure wound therapy (NPWT), hyperbaric oxygen therapy, electrical stimulation, ultrasound, shock wave therapy, or other therapies, may be utilized.

As one of skill in the art will appreciate, such a method is useful to determine that a wound, such as a chronic wound, is healing and that the selected treatment is appropriate. Alternatively, the method is also useful to determine that a wound is not healing, and that the selected treatment may not be appropriate or sufficient, and the wound and/or selected treatment may require further assessment or reconsideration. For example, a non-healing wound may require treatment with an alternative medication, a medication that targets a different infectious agent or an altered dose of anti-microbial agent. An anti-inflammatory agent or altered dose may be required to promote healing. A non-healing wound may require surgical intervention, e.g. surgical debridement to increase blood flow to facilitate wound healing, or may require more frequent dressing changes and/or cleaning to encourage healing. Non-healing may also be evidence of a non-healthy lifestyle, prompting appropriate changes to assist in wound healing, e.g. diet, activity, hygiene or other changes.

The method is also useful to determine whether or not a treatment, such as a newly developed treatment or medication, is useful to treat a wound.

In another aspect of the invention, a kit for use in a method to determine healing status of a wound is provided. The kit comprises a reactant that specifically reacts with a wound biomarker selected from GM-CSF or MMP-13, and optionally, at least one additional reactant that specifically reacts with a second target biomarker, i.e. a biomarker-specific reactant, selected from the group consisting of GM-CSF, MMP-13, albumin, calcium, eotaxin-1, glucose, ICAM-1, IL-6, IL-16, MCP-1, MIP-1α, PDGF-BB and TIMP-4. In one embodiment, the kit comprises a panel of biomarker-specific reactants that target each of GM-CSF, MMP-13, albumin, calcium, eotaxin-1, glucose, ICAM-1, IL-6, IL-16, MCP-1, MIP-1α, PDGF-BB and TIMP-4.

The biomarker-specific reactant, such as an antibody or an antigen-binding fragment thereof, may be bound onto a solid support or panel such as a strip, plate, dipstick, or other support as above described to which fluid from a wound may be applied to enable detection of target biomarkers therein. The kit may also include an implement configured to collect or absorb thereon a sample of fluid from a wound such as a swab, pad, gauze, strip, wipe or cloth. In one embodiment, the reactant is bound directly to an implement configured to collect wound fluid. The reactant is immobilized on the support or implement via any suitable covalent linking means. On exposure to wound fluid, the reactant will complex with target biomarker present in the fluid which may yield a detectable signal, e.g. a colorimetric signal. Alternatively, detection of the biomarker may be accomplished by incubating the biomarker complex with an indicator adapted to link to the biomarker, either directly or via another entity such as an antibody. The indicator may be a detectable label such as fluorescent, phosphorescent or luminescent compound, dye, particle, e.g. colloidal gold or an enzyme label. Preferably, the indicator is one which yields a color change in the presence of target biomarker or other additive which correlates with levels of the biomarker(s) in the wound.

Terms of degree used herein such as “about” refer to a reasonable amount of deviation from a given value such that the end result is not significantly changed, e.g. an amount of deviation of at least +/−5%, and preferably +/−10%.

Embodiments of the invention are described in the following examples which are not to be construed as limiting.

### Example 1

A prospective cohort study was conducted in which wound surface area measurements and wound fluid was collected from VLU patients for 12 weeks, or until the wound was healed. The study was approved by the Government of Western Australia Department of Health South Metropolitan Area Health Service Human Research Ethics Committee. All patients provided written informed consent prior to entry into the study.

Patients were recruited from the leg ulcer clinic at Fremantle Hospital & Health Service in Fremantle, Australia. Eligible patients were male or female over the age of 18 years; had proven evidence of venous disease on photoplethysmography or Duplex scan; a venous leg ulcer greater than 2 cm² in area; ankle brachial indices greater that 0.5; and were able to give informed consent. Patients were excluded if they had an ankle brachial index below 0.5 in order to remove patients with severe arterial disease.

### Wound Area Measurements

Wound area was measured using the Visitrak® (Smith & Nephew, London, UK) digital wound planimetry device. Patients were laid in a comfortable position which allowed for the ulcer to be accessible for measurement. The transparent sterile grid was placed over the ulcer and the outline of the ulcer edge was traced using a permanent marker. The grid was then placed onto the Visitrak® device and the stylus attached to the device was used to re-trace the outline of the ulcer edge. The device then generated the wound area in cm².

The wound area measurements for each wound, each week, were performed three times by two separate raters. It has been previously demonstrated that performing wound area measurements using the Visitrak® three times holds similar reliability of measurement to performing measurements ten times, and has high inter and intra rater reliability. The mean of all six measurements was then used to determine the wound surface area for that week.

### Determination of Healing and Non-Healing Wounds

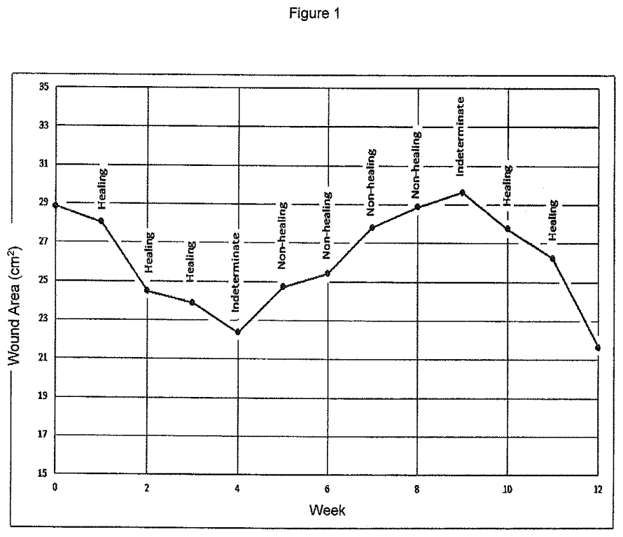

The mean wound surface area each week was used in order to determine if a wound was on a healing or non-healing trajectory. The mean wound surface area for three consecutive weeks was examined (i.e. baseline, week 1, week 2) and the change in wound size from one week to the next was evaluated. If the mean wound area decreased in both weekly segments, the wound was classified as healing for the middle time point. Thus, if the wound area decreased from baseline to week 1, and again from week 1 to week 2, then the wound was classified as healing at week 1 (FIG. 1). If the mean wound surface area increased in both weekly segments, then the wound was classified as non-healing. If there was a decrease in wound surface area in the first weekly segment followed by an increase in the following weekly segment, or vice-versa, then the wound was classified as indeterminate as it is not on a healing or non-healing trajectory at the middle week. This step was then repeated for the next time point (i.e. week 1, week 2, week 3) where the healing status for week 2 was determined. If a wound measurement was missed for a given week, the comparison was carried over to the next week that a wound area measurement was available.

### Wound Fluid Collection

The wound fluid was collected each week by covering the wound with a transparent occlusive dressing for approximately 1 hour. Fluid that had accumulated beneath the occlusive dressing was then aspirated and stored at −80° C. Wound fluid could only be collected on weeks in which the wound was producing exudate, and therefore could not be collected every week for every wound.

### Evaluation of Biomarkers

Wound fluid was analyzed using Multiplex ELISA Assays (RayBiotech, Norcross, Ga., USA) as per manufacturer specifications. The wound fluid was diluted in order to have enough fluid to run two separate ELISA assays and values were corrected for a dilution factor of 2. These assays were used to determine the levels of cytokines, proteases and growth factors in the wound fluid. Standard hospital blood tests were used to evaluate the serum levels of phosphate, lactate, glucose, albumin and calcium each week.

### Wound Treatment

Participants received ulcer treatment that was standard for their ulcer etiology and recommended by the leg ulcer clinic at Fremantle Hospital and Health Service.

### Statistical Analysis

An original sample size of 40 patients was calculated. Independent t-tests were performed between healing and non-healing wounds in order to determine which biomarkers should be placed into the multivariate model. Biomarkers which showed a significance level of <0.1 in the independent t-tests were then included into a multivariate logistic regression model. For variables in the logistic regression model that demonstrated p<0.05 a receiver operating characteristic curve (ROC curve) was created to determine the sensitivity, specificity and the accuracy of the biomarker as a predictor of healing. A cut-off point was determined that could be used to determine healing and non-healing wounds based on the highest Youden's J statistic for points along the ROC curve.

### Results

Patient Demographics—

Forty-five patients were screened for participation in the study, of which 42 were entered (Table 1). The study group consisted of 21 male and 21 female patients with a mean age of 73.1 years. Medical history of patients revealed that 26% of patients had a history of deep vein thrombosis, 21% of patients had diabetes, 19% of patients had either rheumatoid arthritis or another form of auto-immune disorder, and 14% had a history of pulmonary embolism. The mean length of study ulcer was 15.8 months.

TABLE 1

| Characteristic | N = 42 Mean (S.D.) N (%) | |
|---|---|---|
| Age | 73.1 | (11.7) |
| Gender | | |
| Female | 21 | (50%) |
| Male | 21 | (50%) |
| Medical History | | |
| Deep Vein Thrombosis | 11 | (26.2%) |
| Pulmonary Embolism | 6 | (14.3%) |
| Diabetes | 9 | (21.4%) |
| Rheumatoid Arthritis | 4 | (9.5%) |
| Other Autoimmune Disorders | 4 | (9.5%) |
| Ulcer History | | |
| Time Since First Ulcer (Months) | 124.5 | (174.1) |
| Study Ulcer Leg | | |
| Left | 17 | (40.5%) |
| Right | 25 | (59.5%) |
| Length of Study Ulcer (Months) | 15.8 | (26.3) |
| Left | 20.1 | (33.3) |
| Right | 12.9 | (20.6) |
| Clinical Presentations | | |
| Varicose Vein | 30 | (71.4%) |
| Oedema | 28 | (66.7%) |
| Lipodermatosclerosis | 35 | (83.3%) |
| Venous Eczema/Dermatitis | 20 | (47.6%) |
| Type of Compression Therapy | | |
| Low Compression | 0 | (0%) |
| Medium Compression | 27 | (64.3%) |
| High Compression | 15 | (35.7%) |

### Wound Healing Status

There was a total of 105 wound time points in which a surface area measurement, wound fluid collection and blood test were all available for analysis, Of these wound time points, 32 were classified as healing, 27 classified as non-healing and 46 classified as indeterminate.

### Univariate Analysis

The results of the independent t-test univariate analysis are shown in Table 2. In total, 13 of the biomarkers evaluated demonstrated a significant difference between healing and non-healing wounds (p<0.1), while 39 biomarkers did not demonstrate a significant difference between healing and non-healing wounds.

TABLE 2

| Biomarker | Healing (95% CI) | | Non-Healing (95% CI) | | p-value |
|---|---|---|---|---|---|
| Albumin | 23.7 | (21.4-25.9) | 20.2 | (17.8-22.5) | 0.0368 |
| Calcium | 2.11 | (2.00-2.21) | 1.91 | (1.76-2.07) | 0.0347 |
| Eotaxin | 300.0 | (155.7-357.2) | 162.5 | (112.0-212.9) | 0.0006 |
| Glucose | 3.9 | (2.6-5.1) | 1.0 | (0.5-1.5) | <0.0001 |
| GM-CSF | 45.1 | (7.171-83.1) | 187.2 | (137.6-236.8) | <0.0001 |
| ICAM-1 | 120781 | (83858.8-157702) | 196727 | (152885-240569) | 0.0083 |
| IL-6 | 3962.7 | (3702-4223.3) | 3550.5 | (3258.4-3842.7) | 0.0348 |
| IL-16 | 2274.6 | (1378.8-3170.4) | 5155.6 | (3453.4-6857.9) | 0.0038 |
| MCP-1 | 2366 | (2083.7-2648.3) | 2008.8 | (1858.6-2159.0) | 0.0271 |
| MIP-1α | 16519.8 | (11624.3-21415.2) | 24974.7 | (19255.8-30693.6) | 0.0243 |
| MMP-13 | 1192.8 | (802.8-1582.8) | 2301.6 | (1889.1-2714.1) | 0.0002 |
| PDGF-BB | 21.2 | (13.7-28.8) | 8.5 | (2.3-14.6) | 0.0109 |
| TIMP-4 | 1440.1 | (1141.8-1738.3) | 1062.5 | (827.2-1297.9) | 0.047 |
| BLC | 3918.8 | (2900.6-4937.0) | 3534.7 | (2739.9-4329.5) | 0.5539 |
| Eotaxin-2 | 1935.0 | (782.2-2221.9) | 1742.2 | (1423.0-2061.4) | 0.3601 |
| G-CSF | 2999.6 | (2661.8-3387.4) | 3168.7 | (2818.1-3519.4) | 0.5155 |
| I-309 | 456.7 | (298.0-615.5) | 615.3 | (315.4-915.2) | 0.3432 |
| TFN-g | 1.2 | (−1.1-3.5) | 3.1 | (−0.2-6.4) | 0.3411 |
| IL-1a | 1608.9 | (1304.5-1913.3) | 1819.5 | (1366-2273) | 0.4222 |
| IL-1b | 596.7 | (527.3-666.2) | 576.1 | (479.5-672.7) | 0.7285 |
| IL-1ra | 4459.1 | (3813.4-5104.8) | 5118.4 | (4443.4-5793.4) | 0.1545 |
| IL-2 | 66.3 | (10.9-121.8) | 253.3 | (−123.0-629.7) | 0.3213 |
| IL-4 | 13.9149 | (−0.4-28.1) | 3.7 | (2.2-5.2) | 0.1799 |
| IL-5 | 19.2 | (6.8-32.7) | 14.8 | (2.4-27.3) | 0.6304 |
| IL-7 | 12.9 | (9.8-16.1) | 11.4 | (8.6-14.2) | 0.4572 |
| IL-8 | 802.9 | (727.8-878) | 864.2 | (778.9-949.4) | 0.2718 |
| IL-10 | 137.7 | (91.6-183.8) | 156.2 | (107.8-204.6) | 0.574 |
| IL-11 | 938.8 | (562.7-1314.9) | 981.7 | (599.1-1364.3) | 0.871 |
| IL-12p40 | 1170 | (271.4-2068.6) | 534.3 | (335.5-733.0) | 0.1676 |
| IL-12p70 | 2.7 | (0.6-4.7) | 1.4 | (0.7-2.1) | 0.2273 |
| IL-13 | 19.0 | (3.4-34.6) | 49.7 | (−7.4-106.8) | 0.2962 |
| IL-15 | 3.97 | (−2.2-10.1) | 0.007 | (−0.008-0.02) | 0.1984 |
| IL-17 | 150.8 | (86.7382-214.9) | 139.8 | (97.5686-182.0) | 0.7698 |
| MCSF | 22.2 | (5.2-39.2) | 23.5 | (11.6-35.4) | 0.8986 |
| MIG | 17634.9 | (16045.8-19223.9) | 15815.5 | (13308.0-18323.1) | 0.2151 |
| MIP-1b | 486.8 | (438.6-535.1) | 467.7 | (414.3-521.1) | 0.5865 |
| MIP-1d | 8155.1 | (5639.4-10670.9) | 11530.7 | (7548.3-15513.2) | 0.1485 |
| MMP-1 | 400000 | | 400000 | | 1.000 |
| MMP-2 | 8460.3 | (5468.6-11452.0) | 14685.1 | (7577.0-21793.1) | 0.1064 |
| MMP-3 | 82469.9 | (78586.3-86353.5) | 85824.3 | (82299.5-89349.2) | 0.2029 |
| MMP-8 | 32383.2 | (28249.9-36516.5) | 32774.6 | (29275.2-36274.0) | 0.8855 |
| MMP-9 | 91404.8 | (86317.0-96492.7) | 90317.9 | (88675.7-91960.2) | 0.6806 |
| MMP-10 | 6996.6 | (6411.4-7581.8) | 6546.7 | (5870.2-7223.1) | 0.3050 |
| RANTES | 613.9 | (425.7-802.2) | 536.7 | (320.2-753.1) | 0.5817 |
| TIMP-1 | 52031.5 | (48483.5-55579.6) | 53129.8 | (48792.7-57466.8) | 0.6867 |
| TIMP-2 | 11894 | (10793.8-12994.2) | 12492.9 | (11822.0-13163.8) | 0.3574 |
| TNF-a | 1431.3 | (514.7-2347.9) | 2071.2 | (924.5-3217.9) | 0.3704 |
| TNF-b | 0.02 | (−0.012-0.05) | 0 | (0-0) | 0.3253 |
| TNF-RI | 4219.4 | (3627.3-4811.5) | 4368.4 | (4368.4-3705.2) | 0.7319 |
| TNF-RII | 8161.7 | (6978.9-9344.5) | 7205.7 | (6328.9-8082.6) | 0.2003 |
| Phosphate | 1.55 | (1.45-1.65) | 1.48 | (1.34-1.61) | 0.3744 |
| Lactate | 11.19 | (10.60-11.77) | 11.35 | (10.80-11.89) | 0.7033 |

### Multivariable Regression Model

The results of the multivariable regression model showed two biomarkers having significant differences between healing and non-healing wounds, granulocyte macrophage colony stimulating factor (GM-CSF) (p<0.001; odds ratio 126.5) and MMP-13 (v0.004; odds ratio 24.8).

### Receiver Operating Characteristic Curves and Cut-Offs

A receiver operating curve was created for both GM-CSF, MMP-13 and the entire multivariable regression model involving all 13 statistically significant biomarkers. The area under the curve of the entire multivariable model was 0.92 (95% CI 0.58, 0.99), demonstrating a 92% accuracy in discriminating between healing and non-healing wounds. The area under the curve for the receiver operating curve of GM-CSF was determined to be 0.92 (95% CI 0.85, 1.00) demonstrating a 92% accuracy in discriminating between healing and non-healing wounds. MMP-13 showed a 0.78 (95% CI 0.65, 0.90) area under the curve signifying an accuracy of 78% in discriminating between healing and non-healing wounds. The accuracy of the remaining significant biomarkers are as follows: ICAM-1 (0.79; 95% CI 0.68, 0.91), Glucose (0.79; 95% CI 0.67, 0.91), IL-16 (0.77; 95% CI 0.65, 0.89), Eotaxin (0.74; 95% CI 0.62, 0.87), PDGF-BB (0.72; 95% CI 0.58, 0.85), MIP-1a (0.69; 95% CI 0.56, 0.83), TIMP-4 (0.67; 95% CI 0.52, 0.81), IL-6 (0.66; 95% CI 0.51, 0.80), Calcium (0.61; 95% CI 0.46, 0.76), MCP-1 (0.60; 95% CI 0.46, 0.75), and Albumin (0.53; 95% CI0.38, 0.68).

The cut-off value for GM-CSF that demonstrated the highest Youden's statistic was 29.5 pg/ml, which exhibited a sensitivity of 96% and a specificity of 81%. For MMP-13, the cut-off with the highest Youden's J statistic was 962.2 pg/ml which demonstrated a sensitivity of 92% and a specificity of 61%.

### DISCUSSION

The goal of the present study was to evaluate a panel of biomarkers in order to determine if a predictive biomarker of healing exists in the chronic wound fluid. The results of the univariate analysis showed several variables demonstrating significant differences between healing and non-healing wounds, however, upon a multivariable analysis only GM-CSF and MMP-13 demonstrated significant differences between healing and non-healing wounds. Evaluation of ROC curves and cut-off values for both GM-CSF and MMP-13 indicated that GM-CSF provided a 92% accuracy in discriminating between healing and non-healing wounds, and that the cut-off of 29.5 pg/ml exhibited a 96% sensitivity and 81% specificity. In other words, GM-CSF exhibits a high discrimination ability and the cut-off of 29.5 pg/ml demonstrates a 96% accuracy in classifying a non-healing wound as non-healing, and an 81% accuracy in classifying a healing wound as healing.

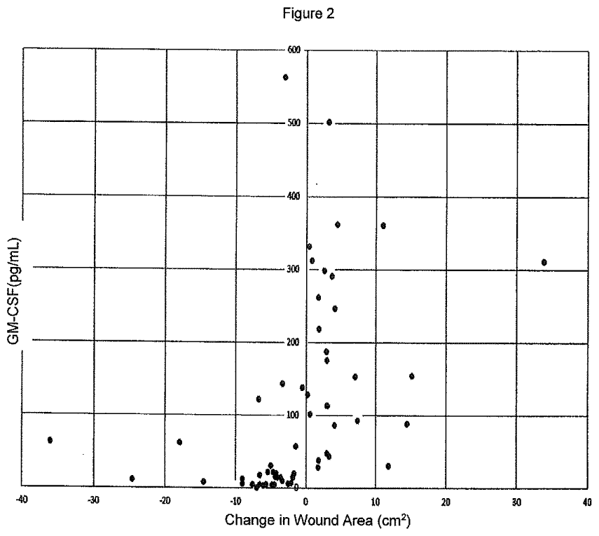

The results of the present study indicate that GM-CSF is elevated in non-healing chronic wounds when compared to healing chronic wounds (FIG. 2).

It has long been identified that chronic non-healing wounds exhibit a prolonged and excessive inflammatory phase characterized by increases in inflammatory cytokines and proteases. While not wishing to be bound by a particular theory, the increase in GM-CSF in chronic wounds seen in this study may account for this excessive inflammatory state, as macrophages which are in the presence of GM-CSF result in an increased inflammatory response through differentiation into M1 macrophages, increased interferon regulatory factor 5 (IRF-5) expression and increased production of inflammatory cytokines TNF-α, IL-1β and IL-6.

The chronic wound environment presents with factors normally key in normal wound healing, such as TNF-α and the MMPs, in excess which contribute to an inability of the wound to heal. While GM-CSF has been shown to be crucial in normal wound healing, the excess seen in chronic wounds may be a key contributing factor to the rises in inflammatory cytokines which in turn result in excessive protease activity and breakdown of the extracellular matrix. This increased protease activity as a result of excessive GM-CSF may include MMP-13, as elevated levels of this protease were related to non-healing wounds. Excessive MMP-13 activity may be responsible for the degradation of growth factors and extracellular matrix linked to non-healing wounds.

The use of GM-CSF for chronic wound healing has previously been investigated, and while appearing promising, the present data indicate that excess GM-CSF is seen in chronic non-healing wounds. In particular, levels of GM-CSF and MMP-13 below threshold levels, are herein shown to be accurate predictive biomarkers of healing, with determined optimal cut-offs which are useful to differentiate between healing and non-healing wounds.

1. A method for determining healing status of a wound comprising:
i) quantifying the expression level of GM-CSF or MMP-13, and optionally one or more additional biomarkers, in a wound tissue sample or wound fluid sample from a wound of a mammalian subject; and
ii) comparing the expression level of GM-CSF or MMP-13 of the wound tissue sample or wound fluid sample to a threshold level and determining that the wound is non-healing if the level of GM-CSF or MMP-13 exceeds the threshold level.

2. The method of claim 1, wherein the expression level of one or more additional biomarkers selected from the group consisting of: GM-CSF, MMP-13, albumin, calcium, eotaxin-1, glucose, ICAM-1, IL-6, IL-16, MCP-1, MIP-1α, PDGF-BB and TIMP-4 is quantified in the wound sample, compared to a threshold level and determined to be indicative of a non-healing wound if the level of the biomarker deviates from the threshold level.

3. The method of claim 2, wherein a determination that the level of any ICAM-1, IL-16 or MIP-1α is greater than their threshold level indicates that the wound is non-healing.

4. The method of claim 2, wherein a determination that the level of any of albumin, calcium, eotaxin-1, glucose, IL-6, MCP-1, PDGF-BB or TIMP-4 is lower than their threshold level indicates that the wound is non-healing.

5. The method of claim 1, wherein the mammalian subject is a human.

6. The method of claim 1, wherein the expression level of GM-CSF or MMP-13, and any additional biomarkers, is quantified using an immunoassay.

7. The method of claim 1, wherein the expression level of GM-CSF or MMP-13, and any additional biomarkers, is quantified by ELISA.

8. The method of claim 1, wherein the expression level of GM-CSF or MMP-13, and any additional biomarkers, is quantified using an antibody microarray.

9. The method of claim 1, wherein the predetermined threshold level of GM-CSF is in the range of about 15-60 pg/μl.

10. The method of claim 1, wherein the predetermined threshold level of MMP-13 is in the range of about 800-1000 pg/ml.

11. The method of claim 1, additionally including the step of treating a non-healing wound.

12. A method of monitoring the effectiveness of a chronic wound treatment in a mammalian subject comprising:
(a) determining the level of GM-CSF or MMP-13, and optionally one or more of additional biomarkers selected from GM-CSF, MMP-13, albumin, calcium, eotaxin-1, glucose, ICAM-1, IL-6, IL-16, MCP-1, MIP-1α, PDGF-BB and TIMP-4, in a first sample from the wound to provide a first biomarker profile;
(b) treating the chronic wound;
(c) determining the level of GM-CSF or MMP-13, and optionally one or more of additional biomarkers selected from GM-CSF, MMP-13, albumin, calcium, eotaxin, glucose, ICAM-1, IL-6, IL-16, MCP-1, MIP-1a, PDGF-BB and TIMP-4, in a second sample from the wound to provide a second biomarker profile; and
(d) comparing the first and second biomarker profiles, wherein a decrease in the level of at least one of GM-CSF or MMP-13, and optionally one or more of the additional biomarkers is indicative of wound healing and indicates that the treatment is effective.

13. A kit for use in a method to determine healing status of a wound, said kit comprising at least one reactant that specifically reacts with GM-CSF and/or MMP-13, and optionally, one or more additional reactants that specifically reacts with a second target biomarker selected from the group consisting of GM-CSF, MMP-13, albumin, calcium, eotaxin-1, glucose, ICAM-1, IL-6, IL-16, MCP-1, MIP-1α, PDGF-BB and TIMP-4.

14. The kit of claim 13, comprising a panel of biomarker-specific reactants that target each of GM-CSF, MMP-13, albumin, calcium, eotaxin-1, glucose, ICAM-1, IL-6, IL-16, MCP-1, MIP-1α, PDGF-BB and TIMP-4.

15. The kit of claim 13, wherein the reactant is an antibody.

16. The kit of claim 13, comprising one or more implements configured to collect a wound sample.

17. The kit of claim 13, comprising a panel on which is bound the at least one or more reactants and which yields a detectable signal in the presence of said biomarker.

18. A kit comprising a plurality of implements configured to collect or absorb thereon a sample of fluid from a wound, and a panel on which is bound at least one reactant specific for a biomarker selected from GM-CSF and MMP-13 which yields a detectable signal in the presence of said biomarker.

19. The kit of claim 18, wherein the implement is a swab, pad, strip or dipstick.

20. The kit of claim 18, wherein the reactant(s) is bound directly to the implement.
